(19) United States
(12) Patent Application Publication
(10) Pub. No.: US 2007/0105882 A1
(43) Pub. Date: May 10, 2007

(54) Polymorphic forms of a known antihyperlipemic agent

(76) Inventors: Simon Nicholas Black, Cheshire (GB); Lianne Frodsham, Cheshire (GB); Nigel Philip Taylor, Cheshire (GB); Kenneth Edwin Herbert Warren, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 10/572,635

(22) PCT Filed: Sep. 17, 2004
(86) PCT No.: PCT/GB04/04133

§ 371 Date: Mar. 17, 2006

(30) Foreign Application Priority Data
Sep. 18, 2003 (GB) 0321827.8

(51) Int. Cl. A61K 31/505; C07D 239/28

(52) U.S. Cl. 514/275; 544/332

(57) ABSTRACT

Two new polymorphic forms of (E)-7-[4-(4-fluorophenyl)-6-isopropyl-2-[methyl(methylsulfonyl)amino]pyrimidin-5-yl](3R,5S)-3,5-dihydroxyhept-6-enoic acid tris(hydroxymethyl)methylammonium salt (1), processes for making them and their use in the production of a pharmaceutical useful in the treatment of, inter alia, hypercholesterolemia, hyperlipoproteinemia and atherosclerosis are described.

This invention concerns new polymorphic forms of (E)-7-[4-(4-fluorophenyl)-6-isopropyl-2-[methyl(methylsulfonyl)amino]pyrimidin-5-yl](3R,5S)-3,5-dihydroxyhept-6-enoic acid tris(hydroxymethyl)methylammonium salt (1) (illustrated below), which is useful for the production of a pharmaceutical useful in the treatment of, inter alia, hypercholesterolemia, hyperlipoproteinemia and atherosclerosis.

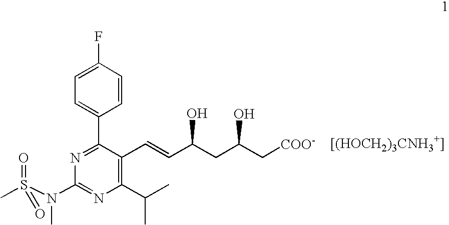

The sodium salt and calcium salt of (E)-7-[4-(4-fluorophenyl)-6-isopropyl-2-[methyl(methylsulfonyl)amino]pyrimidin-5-yl](3R,5S)-3,5-dihydroxyhept-6-enoic acid (hereinafter referred to as compound (2)) were disclosed in European Patent 0521471. This patent also describes a process for the synthesis of the calcium salt, via the sodium salt.

Our International Patent Application WO 00/42024 discloses a crystalline form of the calcium salt of (2), and processes for making it.

Our International Patent Application WO 01/60804 discloses alternative crystalline salts of (2). One of these salts is the tris(hydroxymethyl)methylammonium salt (1). In this application, the process exemplified for formation of tris(hydroxymethyl)methylammonium salt is: acidification of a solution of the methylamine salt of (2) in acetonitrile and water, separation and drying of the organic layer followed by addition of tris(hydroxymethyl)aminomethane at ambient temperature, collection of the crystalline product at ambient temperature and then drying of the crystals at 30° C. under vacuum. This process produces needle shaped crystals of a single polymorph of the salt (1) with an X-ray powder diffraction pattern with peaks at 2-theta=7.9, 8.5, 10.2, 16.7, 18.4, 19.3, 19.8, 20.2, 21.5 and 24.9°.

We have discovered two further polymorphic crystalline forms of the tris(hydroxymethyl)methylammonium salt (1) herein called Forms 2 and 3. Such polymorphic forms may have different solubilities and/or stabilities and/or bioavailabilities and/or different impurity profiles (minor impurities which arise for example because of the process of manufacture and/or isolation) and/or crystal forms which are easier to handle, micronise and/or form into tablets.

According to one aspect of the invention is provided a crystalline tris(hydroxymethyl)methylammonium salt of (E)-7-[4-(4-fluorophenyl)-6-isopropyl-2-[methyl (methylsulfonyl)amino]pyrimidin-5-yl]-(3R,5S)-3,5-dihydroxyhept-6-enoic acid having an X-ray powder diffraction pattern with peaks at 2-theta=3.2, 6.3, 9.5 and 11.0. This crystalline form is hereinafter referred to as Form 2.

According to another aspect of the invention there is provided Form 2 having an X-ray powder diffraction pattern with peaks at 2-theta=3.2, 6.3, 9.5, 11.0, 12.0, 12.4, 13.9 and 21.5.

According to another aspect of the invention is provided Form 2 having an X-ray powder diffraction pattern with peaks at 2-theta=3.2, 6.3, 9.5, 11.0, 12.0, 12.4, 13.9, 15.8, 21.5, 22.7, 23.6 and 24.9.

According to another aspect of the invention is provided Form 2 having an X-ray powder diffraction pattern substantially as shown in FIG. 1.

It will be appreciated that the 2-theta values listed in the aspects of the invention hereinbefore for Form 2, and hereinafter for Form 3, are chosen because they most clearly differentiate one Form from another, although they do not necessarily represent the most intense peaks.

The Form 2 polymorphic salt of this aspect of the invention may be produced by the following process: a sample of amorphous tris(hydroxymethyl)methylammonium salt (1) (which may be prepared by freeze drying an aqueous solution of the salt (1)) is slurried in a suitable organic solvent at a temperature below ambient temperature, the resultant mixture is filtered and the resulting product is dried as necessary.

Suitable organic solvents may be determined experimentally by the skilled person. Conveniently, the organic solvent is acetonitrile, ethyl acetate or MTBE (methylt-butylether).

Conveniently the mixture is slurried for an extended period, for example for 24 hours. Conveniently, the mixture is slurried at a temperature below ambient temperature which is for example, between about 0° C. and 10° C., such as between about 0° C. and 5° C., and preferably at about 0° C.

The product is conveniently dried by prolonged filtration under vacuum, the use of temperatures above ambient temperature preferably being avoided in order to avoid any risk of conversion of polymorphic form.

It will be appreciated that Form 2 may be produced by alternative methods, for example crystallisation from a solution in a suitable organic solvent at low temperature.

According to a further aspect of the invention there is provided a crystalline tris(hydroxymethyl)methylammonium salt of (E)-7-[4-(4-fluorophenyl)-6-isopropyl-2-[methyl(methylsulfonyl)amino]pyrimidin-5-yl]-(3R,5S)-3,5-dihydroxyhept-6-enoic acid having an X-ray powder diffraction pattern with peaks at 2-theta=6.9 and 13.1. This crystalline form is hereinafter referred to as Form 3.

According to a further aspect of the invention there is provided Form 3 having an X-ray powder diffraction pattern with peaks at 2-theta=6.9, 13.1, 14.9 and 20.6.

According to a further aspect of the invention there is provided Form 3 having an X-ray powder diffraction pattern with peaks at 2-theta=6.9, 8.5, 9.0, 13.1, 14.9, 17.2, 18.2, 18.6, 19.0, 19.4, 20.6 and 25.4.

According to another aspect of the invention there is provided Form 3 having an X-ray powder diffraction pattern substantially as shown in FIG. 2.

The Form 3 polymorphic salt of the above aspects of the invention may be produced by the following process: a sample of amorphous tris(hydroxymethyl)methylammonium salt (1) (which may be prepared by freeze drying an aqueous solution of the salt (1)) is slurried in isopropanol at a temperature below ambient temperature, the resultant mixture is filtered and the resulting product is dried.

Conveniently the mixture is slurried for an extended period, for example for 24 hours. Conveniently, the mixture is slurried at a temperature below ambient temperature which is, for example, between about 0° C. and 10° C., such as between about 0° C and 5° C., and preferably at about 0° C.

The product is conveniently dried by prolonged filtration under vacuum, the use of temperatures above ambient temperature preferably being avoided in order to avoid any risk of conversion of polymorphic form.

Thermal Gravimetric Analysis of samples of Form 3 indicates that the polymorphic form is solvated, which arises from the method of manufacture and will be water and/or isopropanol.

Form 2 and Form 3 may also be characterised by any suitable method known in the art.

The X-ray powder diffraction spectra were determined by mounting a sample of the crystalline form on Siemans single silicon crystal (SSC) wafer mounts and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated x-ray source was passed through an automatic variable divergence slit set at V20 (20 mm path length) and the reflected radiation directed through a 2 mm antiscatter slit and a 0.2 mm detector slit. The sample was exposed for 4 seconds per 0.02 degree 2-theta increment (continuous scan mode) over the range 2 degrees to 40 degrees 2-theta in theta-theta mode. The running time was 2 hours 6 minutes and 40 seconds. The instrument was equipped with a scintillation counter as detector. Control and data capture was by means of a DECpc LPv 433sx personal computer running with Diffrac AT (Socabim) software.

It will be understood that the 2-theta values of an X-ray powder diffraction pattern may vary slightly from one machine to another or from one sample to another, and so the values quoted are not to be construed as absolute. It will also be understood that the relative intensities of peaks may vary according to the orientation of the sample under test so that the intensities in the XRD traces included herein are illustrative and not intended to be used for absolute comparison.

Forms 2 and 3 obtained according to the present invention are substantially free from other crystal and non-crystal forms of tris(hydroxymethyl)methylammonium salt of (E)-7-[4-(4-fluorophenyl)-6-isopropyl-2-[methyl(methylsulfonyl)amino]pyrimidin-5-yl]-(3R,5S)-3,5-dihydroxyhept-6-enoic acid. The term “substantially free from other crystal and non-crystal forms” shall be understood to mean that the desired crystal form (Form 2 or Form 3) contains less than 50%, preferably less than 10%, more preferably less than 5% of any other forms of the tris(hydroxymethyl)methylammonium salt of (E)-7-[4-(4-fluorophenyl)-6-isopropyl-2-[methyl (methylsulfonyl)amino]pyrimidin-5-yl]-(3R,5S)-3,5-dihydroxyhept-6-enoic acid.

The utility of the compounds of the invention may be demonstrated by standard tests and clinical studies, including those described in EPA 521471.

According to a further feature of the invention is a method of treating a disease condition wherein inhibition of HMG CoA reductase is beneficial which comprises administering to a warm-blooded mammal an effective amount of Form 2 or Form 3. The invention also relates to the use of Form 2 or Form 3 in the manufacture of a medicament for use in a disease condition.

The compound of the invention may be administered to a warm-blooded animal, particularly a human, in need thereof for treatment of a disease in which HMG CoA reductase is implicated, in the form of a conventional pharmaceutical composition. Therefore in another aspect of the invention, there is provided a pharmaceutical composition comprising Form 2 or Form 3 in admixture with a pharmaceutically acceptable carrier.

Such compositions may be administered in standard manner for the disease condition that it is desired to treat, for example by oral, topical, parenteral, buccal, nasal, vaginal or rectal administration or by inhalation. For these purposes Form 2 or Form 3 may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solution or suspensions or sterile emulsions. A preferred route of administration is oral. Form 2 or Form 3 will be administered to humans at a daily dose in, for example, the ranges set out in EPA 521471. The daily doses may be given in divided doses as necessary, the precise amount of the Form received and the route of administration depending on the weight, age and sex of the patient being treated and on the particular disease condition being treated according to principles known in the art.

According to a further feature of the invention, there is provided a process for the manufacture of a pharmaceutical composition containing Form 2 or Form 3 as active ingredient, which comprises admixing Form 2 or Form 3 together with a pharmaceutically acceptable carrier.

It will be appreciated that Form 2 and Form 3 may be converted to alternative salts of (E)-7-[4-(4-fluorophenyl)-6-isopropyl-2-[methyl(methylsulfonyl)amino]pyrimidin-5-yl]-(3R,5S)-3,5-dihydroxyhept-6-enoic acid, such as the sodium or calcium salt, and the alternative salt may then be used for treatment of a disease in which HMG CoA reductase is implicated, for example as a pharmaceutical composition, as hereinbefore described for Form 2 and Form 3.

Therefore in a further aspect of the invention, there is provided the use of Form 2 or Form 3 as an intermediate in the manufacture of (E)-7-[4-(4-fluorophenyl)-6-isopropyl-2-[methyl(methylsulfonyl)amino]pyrimidin-5-yl]-(3R,5S)-3,5-dihydroxyhept-6-enoic acid calcium salt.

Isolation of a crystalline salt, such as Form 2 or Form 3, allows purification by recrystallisation if necessary. This may be advantageous where, for example, an alternative, non-crystalline salt form is required. Thus a crystalline salt form can be used as a processing aid in the manufacture of non-crystalline salt forms, or crystalline salt forms whose properties are such that purification by recrystallisation is not straightforward. In particular, it is known that the calcium salt of (E)-7-[4-(4-fluorophenyl)-6-isopropyl-2-[methyl(methylsulfonyl)amino]pyrimidin-5-yl]-(3R,5S)-3,5-dihydroxyhept-6-enoic acid is generally amorphous unless crystallised under specific conditions.

In a further aspect of the invention, there is provided the use of Form 2 or Form 3 as a processing aid in the manufacture of (E)-7-[4-(4-fluorophenyl)-6-isopropyl-2-[methyl(methylsulfonyl)amino]pyrimidin-5-yl]-(3R,5S)-3,5-dihydroxyhept-6-enoic acid calcium salt.

The invention is further illustrated, but not limited by the following examples.

### EXAMPLE 1

Amorphous tris(hydroxymethyl)methylammonium salt (1) (1 g), prepared by freeze-drying an aqueous solution of the salt (which may be prepared according to the method described in WO 01/60804), was added to acetonitrile (10 ml) at 0° C. and stirred at 0° C. for 24 h. The slurry was filtered under vacuum to dryness to yield tris(hydroxymethyl)methylammonium salt (1) Form 2.

¹H NMR (d6-DMSO) δ: 1.22 (dd, 6H), 1.36 (m, 1H), 1.52 (m, 1H), 2.07 (m, 1H), 2.19 (m, 1H), 3.37 (s, 6H), 3.45 (s, 3H), 3.55 (s, 3H), 3.76 (m, 1H), 4.21 (q, 1H), 5.54 (dd, 1H), 6.51 (dd, 1H), 7.28 (t, 2H), 7.72 (m, 2H)

### EXAMPLE 2

Amorphous tris(hydroxymethyl)methylammonium salt (1) (1 g), prepared by freeze-drying an aqueous solution of the salt, was added to ethyl acetate (10 ml) at 0° C. and stirred at 0° C. for 24 h. The slurry was filtered under vacuum to dryness to yield tris(hydroxymethyl)methylammonium salt (1) Form 2.

### EXAMPLE 3

Amorphous tris(hydroxymethyl)methylammonium salt (1) (1 g), prepared by freeze-drying an aqueous solution of the salt, was added to MTBE (10 ml) at 0° C. and stirred at 0° C. for 24 h. The slurry was filtered under vacuum to dryness to yield tris(hydroxymethyl)methylammonium salt (1) Form 2.

### EXAMPLE 4

Amorphous tris(hydroxymethyl)methylammonium salt (1) (1 g), prepared by freeze-drying an aqueous solution of the salt, was added to isopropyl alcohol (10 ml) at 0° C. and stirred at 0° C. for 24 h. The slurry was filtered under vacuum to dryness to yield tris(hydroxymethyl)methylammonium salt (1) Form 3.

¹H NMR (d6-DMSO) δ: 1.04 (d, from isopropyl alcohol), 1.22 (dd, 6H), 1.36 (m, 1H), 1.52 (m, 1H), 2.07 (m, 1H), 2.19 (m, 1H), 3.37 (s, 6H), 3.45 (s, 3H), 3.55 (s, 3H), 3.76 (m, 1H), 3.78 (m, from isopropyl alcohol), 4.21 (q, 1H), 5.54 (dd, 1H), 6.51 (dd, 1H), 7.28 (t, 2H 7.72 (m, 2H).

Identity of the samples were confirmed by ¹H NMR. ¹H NMR were analysed using a Bruker DPX400 operating at a field strength of 400 MHz, using d6-dimethylsulfoxide as a solvent. Chemical shifts were measured in parts per million relative to tetramethylsilane. Peak multiplicities are expressed as follows: s=singlet, d=doublet, q=quartet, t=triplet, m=multiplet.

[image]

| 2-theta | d-spacing | Relative Intensity |
|---|---|---|
| 3.2 | 27.8 | 29 |
| 6.3 | 14.0 | 18 |
| 9.5 | 9.3 | 54 |
| 11.0 | 8.0 | 99 |
| 12.0 | 7.4 | 14 |
| 12.4 | 7.2 | 31 |
| 13.9 | 6.4 | 51 |
| 15.8 | 5.6 | 22 |
| 21.5 | 4.1 | 100 |
| 22.7 | 3.9 | 25 |
| 23.6 | 3.8 | 19 |
| 24.9 | 3.6 | 16 |

[image]

| 2-theta | d-spacing | Relative Intensity |
|---|---|---|
| 6.9 | 12.8 | 100 |
| 8.5 | 10.5 | 41 |
| 9.0 | 9.9 | 12 |
| 13.1 | 6.7 | 13 |
| 14.9 | 6.0 | 24 |
| 17.2 | 5.1 | 58 |
| 18.2 | 4.9 | 58 |
| 18.6 | 4.8 | 39 |
| 19.0 | 4.7 | 38 |
| 19.4 | 4.6 | 30 |
| 20.6 | 4.3 | 63 |
| 25.4 | 3.5 | 30 |

1. A crystalline form of the compound tris(hydroxymethyl)methylammonium salt of (E)-7-[4-(4-fluorophenyl)-6-isopropyl-2-[methyl(methylsulfonyl)amino]pyrimdin-5-yl]-(3R,5S)-3,5-dihydroxyhept-6-enoic acid of the formula (I) having an X-ray powder diffraction pattern with specific peaks at 2-theta=3.2, 6.3, 9.5 and 11.0.

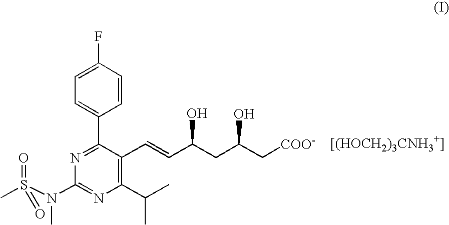

2. A crystalline form as claimed in claim 1 having an X-ray powder diffraction pattern with specific peaks at 2-theta =3.2, 6.3, 9.5, 11.0, 12.0, 12.4, 13.9 and 21.5.

3. A crystalline form as claimed in claim 1 having an X-ray powder diffraction pattern with specific peaks at 2-theta =3.2, 6.3, 9.5, 11.0, 12.0, 12.4, 13.9, 15.8, 21.5, 22.7, 23.6 and 24.9.

4. A crystalline form of the compound tris(hydroxymethyl)methylammonium salt of (E)-7-[4-(4-fluorophenyl)-6-isopropyl-2-[methyl(methylsulfonyl)amino]pyrimidin-5-yl]-(3R,5S)-3,5-dihydroxyhept-6-enoic acid having an X-ray powder diffraction pattern with specific peaks at 2-theta =6.9 and 13.1.

5. A crystalline form as claimed in claim 4 having an X-ray powder diffraction pattern with specific peaks at 2-theta =6.9, 13.1, 14.9 and 20.6.

6. A crystalline form as claimed in claim 4 having an X-ray powder diffraction pattern with specific peaks at 2-theta =6.9, 8.5, 9.0, 13.1, 14.9, 17.2, 18.2, 18.6, 19.0, 19.4, 20.6 and 25.4.

5. A pharmaceutical composition comprising a crystalline form as claimed in any one of the preceding claims, together with a pharmaceutically acceptable carrier.

6. A process for the manufacture of a pharmaceutical composition as claimed in claim 5 which comprises admixing a crystalline form as claimed in claim 1 or claim 4 together with a pharmaceutically acceptable carrier.

7. The use of a crystalline form as claimed in claim 1 or claim 4 in the manufacture of a medicament.

8. A method of treating a disease condition wherein inhibition of HMG CoA reductase is beneficial which comprises administering to a warm-blooded mammal an effective amount of a crystalline form as claimed in claim 1 or claim 4.

9. A process for the manufacture of a crystalline form as claimed in claim 1 or claim 4 which comprises forming crystals by:
a) slurrying a sample of amorphous tris(hydroxymethyl)methylammonium salt (1) in an organic solvent at a temperature below ambient temperature;
b) filtration of the resultant mixture; and
c) drying of the resultant product as necessary.

10. A process as claimed in claim 9 for the manufacture of Form 2 wherein the organic solvent is acetonitrile, ethyl acetate or MTBE (methylt-butylether).

11. A process for the manufacture of a crystalline form as claimed in claim 9 for the manufacture of Form 3 wherein the organic solvent is isopropanol.

12. A process as claimed in any one of claims 9 to 11 wherein the temperature is about 0° C.
